(19) United States
(12) Patent Application Publication
(10) Pub. No.: US 2025/0339385 A1
(43) Pub. Date: Nov. 6, 2025

(54) TOPICAL FORMULATION OF DIMETHYLCURCUMIN

(71) Applicant: ANNJI PHARMACEUTICAL CO., LTD., TAIPEI CITY (TW)

(72) Inventors: CHAN-JUNG LI, NEW TAIPEI CITY (TW); SHIN-YU LAI, NEW TAIPEI CITY (TW); GUAN HOCK KHOO, NEW TAIPEI CITY (TW)

(73) Assignee: ANNJI PHARMACEUTICAL CO., LTD., TAIPEI CITY (TW)

(21) Appl. No.: 18/564,518

(22) PCT Filed: Jul. 1, 2022
(86) PCT No.: PCT/US2022/036018

§ 371 Date: Nov. 27, 2023

Related U.S. Application Data
(60) Provisional application No. 63/218,153, filed on Jul. 2, 2021.

(51) Int. Cl. A61K 31/12; A61K 9/06; A61K 47/10; A61K 47/12; A61K 47/14; A61K 47/34

(52) U.S. Cl. CPC A61K 31/12; A61K 9/06; A61K 47/10; A61K 47/12; A61K 47/14; A61K 47/34

(57) ABSTRACT

Provided is a pharmaceutical composition that may be suitable for topical application. The pharmaceutical composition for topical application includes dimethylcurcumin and/or a salt thereof as an active pharmaceutical ingredient in a range of from 0.001% w/w to 0.2% w/w. The pharmaceutical composition for topical application may further include an oil solvent system in an amount of at least 8% w/w.

### CROSS-REFERENCE TO RELATED APPLICATIONS

This application is a U.S. National Stage Application under 35 U.S.C. § 371 of International Application No. PCT/US2022/036018 which was filed on 1 Jul. 2022. This application claims priority to and the benefit of U.S. 63/218,153 filed 2 Jul. 2021, the disclosures of which are incorporated in their entirety by reference herein.

### BACKGROUND

Curcuminoids include curcumin and derivatives thereof. These compounds may be synthesized in a laboratory or they may be obtained in nature. A common natural source of curcuminoids is from ginger root, such as Curcuma longa. Curcuminoids may be used as ingredients in the food, dietary supplement, and cosmetic industries. In some instances, curcuminoids may provide coloring and flavoring in industrial formulations. More recently, curcuminoids have been explored for use in the pharmaceutical industry.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a composition of a pharmaceutical composition suitable for topical application, comprising a curcuminoid compound of Formula I and/or a salt thereof as an active pharmaceutical ingredient (API) in a range from 0.001% w/w to 0.2% w/w, and an oil solvent system in an amount of at least 8% w/w, wherein % w/w is compared to the overall weight of the pharmaceutical composition.

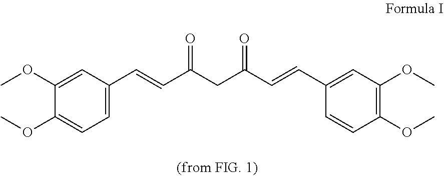

Other aspects and advantages of the claimed subject matter will be apparent from the following description and the appended claims.

### BRIEF DESCRIPTION OF DRAWINGS

FIG. 1 shows a diagram of chemical formula, Formula I according to one or more embodiments of the present disclosure.

FIG. 2 shows a photograph of Formulation 2 (F2) under a polarized light microscope according to one or more embodiments of the present disclosure.

FIG. 3 shows a photograph of Formulation 3 (F3) under a polarized light microscope according to one or more embodiments of the present disclosure.

FIG. 4 shows a photograph of Formulation 4 (F4) under a polarized light microscope according to one or more embodiments of the present disclosure.

FIG. 5 shows a photograph of Formulation 5 (F5) under a polarized light microscope according to one or more embodiments of the present disclosure.

FIG. 6 shows a photograph of Formulation 6 (F6) under a polarized light microscope according to one or more embodiments of the present disclosure.

FIG. 7 shows a photograph of Formulation 7 (F7) under a polarized light microscope according to one or more embodiments of the present disclosure.

FIG. 8 shows a photograph of Formulation 8 (F8) under a polarized light microscope according to one or more embodiments of the present disclosure.

FIG. 9 shows a photograph of Formulation 9 (F9) under a polarized light microscope according to one or more embodiments of the present disclosure.

FIG. 10 shows a photograph of Formulation 10 (F10) under a polarized light microscope according to one or more embodiments of the present disclosure.

FIG. 11 shows a photograph of Formulation 11 (F11) under a polarized light microscope according to one or more embodiments of the present disclosure.

FIG. 12 shows a photograph of Formulation 12 (F12) under a polarized light microscope according to one or more embodiments of the present disclosure.

FIG. 13A shows a photograph of Formulation 13 (F13) under a polarized light microscope according to one or more embodiments of the present disclosure.

FIG. 13B shows a higher resolution (zoomed in) version of the inset marked in FIG. 13A, which is a photograph of F13 under a polarized light microscope according to one or more embodiments of the present disclosure.

FIG. 14 shows a photograph of Formulation 14 (F14) under a polarized light microscope according to one or more embodiments of the present disclosure.

FIG. 15 shows a photograph of Formulation 15 (F15) under a polarized light microscope according to one or more embodiments of the present disclosure.

FIG. 16 shows an HPLC chromatogram of Formulation 7 (F7) according to one or more embodiments of the present disclosure.

FIG. 17 shows an HPLC chromatogram of Formulation 14 (F14) according to one or more embodiments of the present disclosure.

FIG. 18 shows an HPLC chromatogram of Formulation 15 (F15) according to one or more embodiments of the present disclosure.

FIG. 19 shows a chromatograph of a standard solution including API, as a control.

FIG. 20 shows a chromatograph of a blank with solvent/diluent only.

FIG. 21 shows a photograph of sample D1 under a polarized light microscope according to one or more embodiments of the present disclosure.

FIG. 22 shows a photograph of sample D2 under a polarized light microscope according to one or more embodiments of the present disclosure.

FIG. 23 shows a photograph of sample D3 under a polarized light microscope according to one or more embodiments of the present disclosure.

FIG. 24 shows a photograph of sample D4 under a polarized light microscope according to one or more embodiments of the present disclosure.

FIG. 25 shows a photograph of sample D5 under a polarized light microscope according to one or more embodiments of the present disclosure.

### DETAILED DESCRIPTION

Specific embodiments of the disclosure will now be described in detail with reference to the accompanying figures. In the following detailed description of one or more embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Embodiments disclosed herein relate generally to a pharmaceutical composition. The pharmaceutical composition is a formulation suitable for a topical application.

Embodiments disclosed herein relate generally to a method of preparing a pharmaceutical composition.

### Pharmaceutical Composition

The pharmaceutical composition is a formulation containing an active pharmaceutical ingredient (API) and an oil solvent system, such an ointment or a cream, suitable for use as a topical application.

In one or more embodiments, the pharmaceutical composition includes an API, an oil solvent system, a surfactant system, an aqueous system, and other components.

The pharmaceutical composition is a matrix to carry the API, such that the API is delivered in a topical manner to the skin, and in a therapeutically effective and stable amount. A therapeutically effective and stable amount of the API is a % w/w range that the API is included in the weight of the overall composition, to be described.

The term “therapeutically effective amount” as used herein refers to the amount of a compound or composition that, when administered to a patient for treating a disease or disorder, is sufficient to affect such treatment for the disease or disorder. However, in addition to being therapeutically effective, the amount of API present in the claimed composition is also stable.

For the pharmaceutical composition to deliver the API in stable amount, the API has sufficient solubility. Sufficient solubility is an even distribution of the API (by weight and by concentration) throughout the pharmaceutical composition, where the overall pharmaceutical composition is a cream having evenly distributed organoleptic properties (including but not limited to texture, smoothness, and color). For example, the overall pharmaceutical composition is without phase separation.

Further, the pharmaceutical composition provides API stability over a period of time under certain standard storage conditions. Stability of the API includes chemical stability, physical stability, or a combination thereof. Physical stability means that crystal formation, precipitation, or other agglomeration of the API is prevented, such that sufficient solubility is maintained over such period of time. Chemical stability means that the API retains its chemical configuration and retains its potency in the pharmaceutical composition over a period of time. The period of time that stability and sufficient solubility of a pharmaceutical composition is maintained is typically up to 5 years, such as 5 years, 4 years, 3 years, 2 years, 1 year, 11 months, 10 months, 9 months, 8 months, 7 months, 6 months, 5 months, 4 months, 3 months, 2 months, or 1 month. Such a period of time may be projected under accelerated testing conditions that typically are of higher temperature and humidity than its standard storage conditions. These accelerated testing conditions may sometimes be referred to as stability tests or stability testing, but stability tests/testing are not limited to accelerated testing conditions.

To provide sufficient solubility and stability for the API, the pharmaceutical composition itself is stable. Stability of the pharmaceutical composition is defined by maintaining its original organoleptic properties and allowing minimal degradation over a period of time. Minimal degradation may include limiting the formation of impurities over time. For example, overall impurities may be limited to 10% or less, 9% or less, 8% or less, 7% or less, 6% or less, 5% or less, 4% or less, 3% or less, 2% or less, or 1% or less of the overall pharmaceutical composition. Impurities include molecules that are not present or native to the ingredients included in the pharmaceutical composition. Examples of these impurities are commonly known in the art, and may vary based on ingredients and ingredient concentrations in a formulation. Impurities may arise from known degradation processes such as oxidation, radical reactions (from oxidation, UV radiation, or other radical source), esterification, saponification, additions, substitutions, combinations thereof, and the like.

The period of time that the pharmaceutical composition is stable is the same period of time that stability and sufficient solubility of the API is maintained, such as up to 5 years, such as 5 years, 4 years, 3 years, 2 years, 1 year, 11 months, 10 months, 9 months, 8 months, 7 months, or 6 months. Under accelerated testing conditions, the period of time may be shortened to up to months, such as 12 months, 6 months, 3 months, 4 weeks, 14 days, 10 days, 6 days, 5 days, 4 days, 3 days, 2 days or 1 day.

Thus, the pharmaceutical composition is stable and provides sufficient solubility for the API, which further provides API stability.

The definition of “stability testing” is as follows, referenced from Bajaj, et al. (Bajaj et al., “Stability Testing of Pharmaceutical Products,” JAPS online, 02 (03), 2012, pp. 129-138, world wide web address “japsonline.com/admin/php/uploads/409_pdf.pdf”): Stability testing is termed as a complex process because of involvement of a variety of factors influencing the stability of a pharmaceutical product. These factors include stability of the active ingredient(s); interaction between active ingredients and excipients, manufacturing process followed, type of dosage form, container/closure system used for packaging and light, heat and moisture conditions encountered during shipment, storage and handling.

In one or more embodiments, “stability” means the integrity of API, the solubility, interaction of API and excipients etc. under a given standard storage condition. The “integrity of API” is defined by the quantity of impurities under the standard storage condition.

The long term stability is projected through an accelerated testing condition, for example 37° C. for 3 months, 37° C. for 30 days, 37° C. for 28 days, 37° C. for 14 days, 50° C. for 20 days, 50° C. for 17 days, 50° C. for 15 days, 50° C. for 10 days, 50° C. for 6 days, or 50° C. for 1 day. The “unsatisfactory stability” may be defined as API insolubility in the excipients, loss of API integrity and/or the presence of impurities that exceed certain acceptable range.

An acceptable range of API solubility is no more than 0.2% in the pharmaceutical composition (API/pharmaceutical composition) in the presence of aqueous solvent.

Generally, an API is a substance or mixture of substances intended to be used in the manufacture of a drug product, when used in the production of a drug, becomes an active ingredient in the drug product.

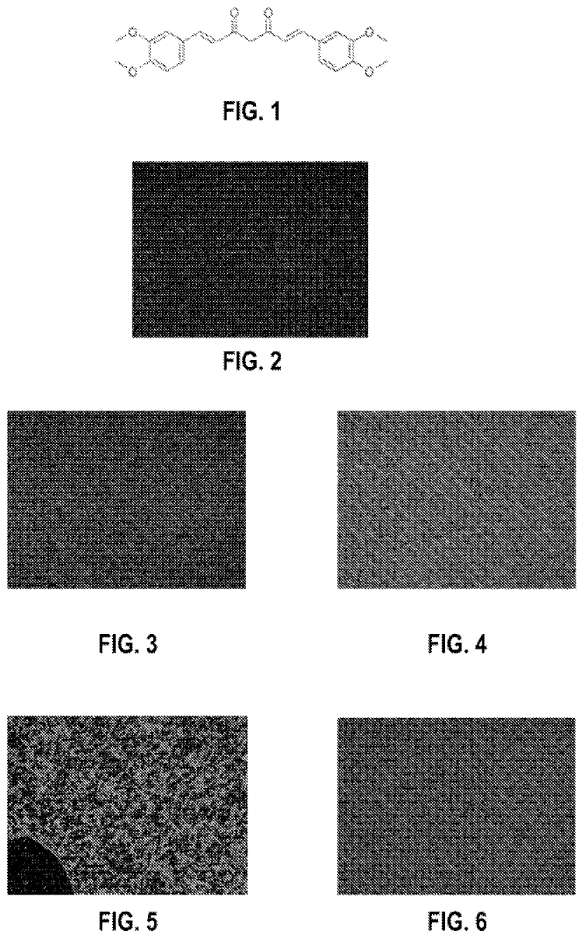

In one or more embodiments, the API is a curcuminoid compound called “dimethylcurcumin,” as shown in FIG. 1 (Formula I).

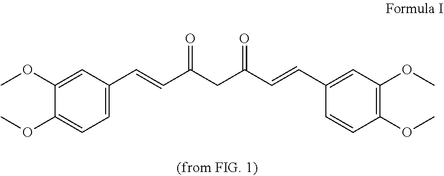

As used herein, the term “Formula” refers to the API and the term “Formulation(s)” (and “formulation(s)”) refer to one or more composition that contains the API.

The API may be in a range with a lower limit of any of 0.001% w/w, 0.005% w/w, 0.01% w/w, 0.02% w/w, 0.03% w/w, 0.04% w/w, 0.05% w/w, 0.06% w/w, 0.07% w/w, 0.08% w/w, or 0.09% w/w, and an upper limit of any of 0.1% w/w, 0.15% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, or 5% w/w, where any lower limit can be used in combination with any upper limit.

The recitation “% w/w” is defined as the weight percentage of the component compared to the weight percentage of the overall pharmaceutical composition (known as weight percent or percent weight by weight).

The oil solvent system is a mixture of substances that contribute to an oil phase of the pharmaceutical composition. The oil solvent system may include an ester, an organic alcohol, a glycol ether, an organosilicon, or any combination thereof.

In one or more embodiments, the oil solvent system is in an amount of at least 8% w/w.

The oil solvent system may be in a range with a lower limit of any of 4% w/w, 4.5% w/w, 5% w/w, 5.5% w/w, 6% w/w, 6.5% w/w, 7% w/w, 7.5% w/w, 8% w/w, 8.5% w/w, 9.0% w/w, 9.5% w/w, 10% w/w, 10.5% w/w, 11% w/w, 11.5% w/w, 12% w/w, or 12.5% w/w, and an upper limit of any of 13% w/w, 14% w/w, 15% w/w, 16% w/w, 17% w/w, 18% w/w, 19% w/w, 20% w/w, 21% w/w, 22% w/w, 23% w/w, 24% w/w, 25% w/w, 26% w/w, 27% w/w, 28% w/w, 29% w/w, 30% w/w, 31% w/w, 32% w/w, 33% w/w, 34% w/w, 35% w/w, 36% w/w, 37% w/w, 38% w/w, 39% w/w, 40% w/w, where any lower limit can be used in combination with any upper limit.

In the pharmaceutical composition, the oil solvent system and API have a weight ratio (oil solvent system: API) called a “solvent-to-API ratio.” In one or more embodiments, the solvent-to-API ratio is more than 82:1, more than 84:1, more than 86:1, more than 88:1, more than 90:1, more than 92:1, more than 94:1, more than 96:1, more than 98:1, more than 100:1, more than 102:1, more than 104:1, more than 106:1, more than 108:1, more than 110:1, more than 112:1, more than 114:1, more than 116:1, more than 118:1, more than 120:1, more than 122:1, more than 124:1, more than 126:1, more than 128:1, more than 130:1, more than 135:1, more than 136:1, more than 155:1, more than 157:1, more than 163:1, more than 179:1, more than 239:1, more than 270:1, or more than 435:1.

The ester may be one or more organic compound that includes 15 to 20 carbons from either or both of the acid and alcohol forming the ester. In one or more embodiments, the ester includes a single ester group. An example of a suitable ester includes but is not limited to isopropyl myristate (IPM) (CAS Number 110-27-0, available from First Chemical Works, Zhongzheng District, Taipei City, Taiwan, R.O.C.).

The ester, such as IPM, may be in a range with a lower limit of any of 2% w/w, 2.5% w/w, 3% w/w, 3.5% w/w, 4% w/w, 4.5% w/w, 5% w/w, 5.5% w/w, or 6% w/w, and an upper limit of any of 13% w/w, 15% w/w, 17% w/w, 19% w/w, 21% w/w, 23% w/w, 25% w/w, 27% w/w, 29% w/w, 31% w/w, or 33% w/w, where any lower limit can be used in combination with any upper limit.

The organic alcohol may be one or more compound having a benzylic alcohol, an aliphatic alcohol, another suitable alcohol, or a mixture thereof. The organic alcohol may be in any range with a lower limit of 0.05% w/w or greater and with an upper limit of 10% w/w or less.

In one or more embodiments, the organic alcohol includes a compound that has a benzylic alcohol. A benzylic alcohol may be one or more compound that includes an alcohol at a benzylic position of a compound. An example of a suitable benzylic alcohol is benzyl alcohol (CAS Number 100-51-6, available from ACROS Organics, Fair Lawn, New Jersey, U.S.A.).

The benzylic alcohol, such as benzyl alcohol, may be in a range with a lower limit of any of 0.4% w/w, 0.6% w/w, or 0.8% w/w, 1.0% w/w, 1.2% w/w, 1.4% w/w, 1.6% w/w, 1.8% w/w, 2.0% w/w, 2.2% w/w, or 2.4% w/w, and an upper limit of any of 2.6% w/w, 2.8% w/w, 3.0% w/w, 3.2% w/w, 3.4% w/w, 3.6% w/w, 3.8% w/w, 4.0% w/w, 4.2% w/w, 4.4% w/w, 4.6% w/w, 4.8% w/w, or 5.0% w/w, where any lower limit can be used in combination with any upper limit.

In one or more embodiments, the organic alcohol includes a compound that is an aliphatic alcohol. An aliphatic alcohol may be one or more compound that includes 4 to 26 carbons and a terminal or internal alcohol functional group. An example of a suitable aliphatic alcohol includes but is not limited to cetostearyl alcohol (CAS Number 67762-27-0, available from First Chemical Works, Zhongzheng District, Taipei City, Taiwan, R.O.C.).

The aliphatic alcohol, such as cetostearyl alcohol, may be in a range with a lower limit of any of 1% w/w, 1.5% w/w, 2% w/w, 2.5% w/w, 3% w/w, 3.5% w/w, 4% w/w, or 4.5% w/w, and an upper limit of any of 6% w/w, 6.5% w/w, 7% w/w, 7.5% w/w, or 8% w/w.

The glycol ether may be an alkyl ether of ethylene glycol. In one or more embodiments, the glycol ether may be an alkyl ether of a polyoxyethylene, including diethylene glycol.

An example of a suitable glycol ether includes but is not limited to diethylene glycol monoethyl ether (CAS Number 111-90-0, available from Alfa Aesar, Ward Hill, Massachusetts, U.S.A.). Another source of diethylene glycol monoethyl ether is under the trade name Transcutol® P (Gattefossé, Saint-Priest Cedex, Lyon, France).

The glycol ether, such as diethylene glycol monoethyl ether, may be less than 10% w/w, less than 9% w/w, less than 8% w/w, less than 7% w/w, less than 6% w/w, less than 5% w/w, less than 4% w/w, less than 3% w/w, less than 2% w/w, or less than 1% w/w, such as in a range with a lower limit of any of 0.1% w/w, 0.15% w/w, 0.2% w/w, 0.25% w/w, 0.3% w/w, 0.35% w/w, 0.4% w/w, or 0.45% w/w, and an upper limit of any of 0.6% w/w, 0.7% w/w, 0.8% w/w, 0.9% w/w, 1% w/w, 1.5% w/w, 2% w/w, 2.5% w/w, 3% w/w, 3.5% w/w, 4% w/w, 4.5% w/w, 5% w/w, where any lower limit can be used in combination with any upper limit.

In one or more embodiments, the diethylene glycol monoethyl ether is less than 10% w/w, less than 9% w/w, less than 8% w/w, less than 7% w/w, less than 6% w/w, less than 5% w/w, less than 4% w/w, less than 3% w/w, less than 2% w/w, less than 1% w/w, less than 0.6% w/w, less than 0.5% w/w, less than 0.5%, or less than 0.3% w/w.

The organosilicon may be one or more compound including an organosilicon functional group. In one or more embodiments, the organosilicon is a polymeric organosilicon.

An example of a suitable organosilicon includes but is not limited to dimethicone (such as CAS Number 9016-00-6, available from First Chemical Works, Zhongzheng District, Taipei City, Taiwan, R.O.C., but not limited thereto).

The organosilicon, such as dimethicone, may be present at less than 5% w/w, less than 4.5% w/w, less than 4% w/w, less than 3.5% w/w, less than 3% w/w, less than 2.5% w/w, less than 2% w/w, less than 1.5% w/w, less than 1%, or less than 0.5%. For example, the organosilicon may be in a range having a lower limit of any of 0.01% w/w, 0.05% w/w, 0.1% w/w, 0.15% w/w, 0.2% w/w, 0.25% w/w, 0.3% w/w, 0.35% w/w, 0.4% w/w, 0.45% w/w, 0.50% w/w, 0.55% w/w, 0.60% w/w, 0.65% w/w, 0.70% w/w, 0.75% w/w, 0.80% w/w, 0.85% w/w, 0.90% w/w, 0.95% w/w, or 1.00% w/w and an upper limit of any of 1.20% w/w, 1.25% w/w, 1.30% w/w, 1.35% w/w, 1.40% w/w, 1.45% w/w, 1.50% w/w, 2.00% w/w, 2.50% w/w, 3.00% w/w, 3.50% w/w, 4.00% w/w, 4.50% w/w, or 5.00% w/w, where any lower limit can be used in combination with any upper limit.

In the pharmaceutical composition, the oil solvent system as a whole and the dimethicone have a weight ratio (oil solvent system:dimethicone) called a “solvent-to-dimethicone ratio.” In one or more embodiments, the solvent-to-dimethicone ratio is more than 2.3:1, more than 2.4:1, more than 2.5:1, more than 2.6:1, more than 2.7:1, more than 2.8:1, more than 2.9:1, more than 3:1, more than 4:1, more than 5:1, more than 6:1, more than 7:1, more than 8:1, more than 9:1, more than 10:1, more than 11:1, more than 12:1, more than 13:1, more than 14:1, more than 15:1, more than 16:1, more than 17:1, more than 18:1, more than 19:1, more than 20:1, more than 21:1, more than 22:1, more than 23:1, more than 24:1, or more than 25:1, more than 56:1, more than 100:1, or more than 126.2:1.

The surfactant system is a mixture of substances that contribute to the lowering of the surface tension between the oil solvent system and the aqueous system. The surfactant system may include a non-ionic surfactant, a fatty acid, or a combination thereof.

The surfactant system may be in an amount of 16% w/w or less, or less than 16% w/w compared to the weight of the overall composition. For example, the surfactant system may be in a range with a lower limit of any of 1% w/w, 1.2% w/w, 1.4% w/w, 1.6% w/w, 1.8% w/w, 2.0% w/w, 2.2% w/w, 2.4% w/w, 2.6% w/w, 2.8% w/w, 3.0% w/w, 3.2% w/w, or 3.4% w/w, and an upper limit of any of 4.0% w/w, 4.5% w/w, 5.0% w/w, 5.5% w/w, 6.0% w/w, 6.5% w/w, 7.0% w/w, 7.5% w/w, 8.0% w/w, 8.5% w/w, 9.0% w/w, 9.5% w/w, 10.0% w/w, 10.5% w/w, 11.0% w/w, 11.5% w/w, 12.0% w/w, 12.5% w/w, 13.0% w/w, 13.5% w/w, 14.0% w/w, 14.5% w/w, 15.0% w/w, 15.5% w/w, or 16% w/w, compared to the weight of the overall composition, where any lower limit can be used in combination with any upper limit.

The surfactant system may include a non-ionic surfactant. The non-ionic surfactant may be compounds that have one or more functional group including but not limited to an ester, an ether, an alcohol, an acid, an olefin, or combinations thereof.

The non-ionic surfactant may include one or more of a polyethylene glycol ether of cetearyl alcohol, a polyoxyethylene alkyl ether, a polyethylene glycol ether of cholesterol, a polyoxyethylene ether of lanolin alcohol, an ethoxylated methyl glucoside, an ethoxylated alkyl phenol, a polyethylene glycol ether of oleyl alcohol, a polyoxyethylene-polyoxypropylene block copolymer, a polyoxyethylene fatty acid ester, a polyoxyl glyceryl stearate, a polyoxyethylene sorbitan monolaurate, a polyoxyethylene stearyl ether, a polydimethyl siloxane, a methyl glucose poly-ester, methyl glucose poly-ether, a polyethylene glycol derivative of a castor oil, a polyethylene glycol derivative of a fatty acid ester, or a polyethylene glycol derivative of an alcohol ether.

When the non-ionic surfactant is a polyethylene glycol ether of cetearyl alcohol, it may include but is not limited to one or more of ceteareth-12, ceteareth-15, ceteareth-20, ceteareth-30, and cetearyl alcohol/ceteareth-20.

When the non-ionic surfactant is a polyoxyethylene alkyl ether, it may include but is not limited to one or more of ceteth-2, ceteth-10, ceteth-20, and ceteth-23.

When the non-ionic surfactant is a polyethylene glycol ether of cholesterol, it may include but is not limited to one or more of choleth and choleth-24.

When the non-ionic surfactant is a polyoxyethylene ether of lanolin alcohol, it may include but is not limited to one or more of laneth, ethoxylated lanolin, and PEG-75 lanolin.

When the non-ionic surfactant is an ethoxylated methyl glucoside, it may include but is not limited to one or more of methyl gluceth-10 and methyl gluceth-20.

When the non-ionic surfactant is an ethoxylated alkyl phenol, it may include but is not limited to one or more of octoxynol-9 and oxtoxynol-40.

When the non-ionic surfactant is a polyethylene glycol ether of oleyl alcohol, it may include but is not limited to one or more of oleth-2, oleth-5, oleth-10, oleth-20, and oleth-10/oleth-5.

When the non-ionic surfactant is a polyoxyethylene-polyoxypropylene block polymer, it may include but is not limited to one or more of poloxamer 124, poloxamer 182, and poloxamer 407.

When the non-ionic surfactant is a polyoxyethylene fatty acid ester, it may include but is not limited to one or more of PEG-8 laurate, PEG-5 oleate, PEG-26 oleate, PPG-26 oleate, PEG-6 isostearate, polyoxyl distearate, PEG-2 stearate, polyoxyl stearate, pegoxol 7 stearate, PEG-8 stearate, polyoxyl 40 stearate, PEG 6-32 stearate, and PEG-100 stearate.

For example, a suitable surfactant in the surfactant system is polyoxyl 40 stearate, CAS Number 9004-99-3 (reagent grade, available from Tokyo Chemical Industry, Tokyo, Japan; or National Formulary (NF) grade, available from Spectrum Chemical Mfg. Corp., New Brunswick, NJ, USA).

When the non-ionic surfactant is a polyoxyl glyceryl stearate, it may include but is not limited to one or more of PEG-120 glyceryl stearate, polyoxyl glyceryl stearate, and stearoyl polyoxyl glycerides.

When the non-ionic surfactant is a polyoxyethylene sorbitan monolaurate, it may include but is not limited to one or more of polysorbate 20, polysorbate 40, polysorbate 60, and polysorbate 80.

For example, a suitable surfactant in the surfactant system is polysorbate 20, CAS Number 9005-64-5 (extra pure grade), available from Showa Chemical Co., Ltd. (Tokyo, Japan). Another suitable surfactant is polysorbate 80, CAS Number 9005-65-6 (reagent grade), available from Acros Organics (Geel, Belgium).

When the non-ionic surfactant is a polyoxyethylene stearyl ether, it may include but is not limited to one or more of PPG-11 stearyl ether, PPG-15 stearyl ether, steareth-2, steareth-10, steareth-21, and steareth-40.

When the non-ionic surfactant is a polydimethyl siloxane, it may include but is not limited to one or more of PEG/PPG-18/18 dimethicone.

When the non-ionic surfactant is a methyl glucose poly-ester and/or methyl glucose poly-ether, it may include but is not limited to one or more of PEG-120 methyl glucose dioleate, PEG-20 methyl glucose sesquistearate, and PPG-20 methyl glucose ether distearate.

When the non-ionic surfactant is a polyethylene glycol derivative of castor oil, a fatty acid ester, or an alcohol ether, it may include but is not limited to one or more of polyoxyl 35 castor oil, polyoxyl 40 hydrogenated castor oil, PEG-60 hydrogenated castor oil, PEG-20 sorbitan isostearate, PEG-25 propylene glycol stearate, PPG-20 methyl glucose ether distearate, polyoxyl 15 hydroxystearate, polyoxyl 6, polyoxyl 32, palmitostearate, apricot kernel oil PEG-6 esters, polyoxyl 20 cetostearyl ether, and trideceth-10, C13-14 isoparaffin/laureth-7/polyacrylamide.

In one or more embodiments, the non-ionic surfactant is in an amount less than 15% w/w, less than 14% w/w, less than 13% w/w, less than 12% w/w, less than 11% w/w, less than 10.5% w/w, less than 10% w/w, less than 9.5% w/w, less than 9% w/w, less than 8.5% w/w, less than 8.0% w/w, less than 7.5% w/w, less than 7% w/w, less than 6.5% w/w, less than 6% w/w, less than 5.5% w/w, less than 5% w/w, less than 4.5% w/w, less than 4% w/w, less than 3.5% w/w, or less than 3% w/w.

An example of a suitable non-ionic surfactant that may be used is polyoxyl 40 stearate (CAS Number 9004-99-3, available from TCI, Tokyo Chemical Industry Co. Ltd., Tokyo, Japan). In one or more embodiments, polyoxyl 40 stearate, polysorbate 20, polysorbate 80, or other non-ionic surfactant, is in a range with a lower limit of any of 0.8% w/w, 1% w/w, 1.2% w/w, 1.4% w/w, 1.6% w/w, 1.8% w/w, 2.0% w/w, 2.2% w/w, or 2.4% w/w, and an upper limit of any of 2.6% w/w, 2.8% w/w, 3.0% w/w, 3.2% w/w, 3.4% w/w, 3.6% w/w, 3.8% w/w, 4.0% w/w, 4.2% w/w, 4.4% w/w, 4.6% w/w, 4.8% w/w, 5.0% w/w, 5.5% w/w, 6.0% w/w, 6.5% w/w, 7.0% w/w, 7.5% w/w, or 8.0% w/w, where any lower limit can be used in combination with any upper limit.

The surfactant system may include a fatty acid. The fatty acid may include one or more fatty acid molecule having 8 to 26 carbons and a carboxylic acid functional group. The fatty acid may have another functional group, including but not limited to an olefin, an alcohol, or a combination thereof.

In one or more embodiments, the fatty acid is in an amount less than 10% w/w, less than 9.5% w/w, less than 9% w/w, less than 8.5% w/w, less than 8% w/w, less than 7.5% w/w, less than 7% w/w, less than 6.5% w/w, less than 6% w/w, less than 5.5% w/w, less than 5% w/w, less than 4.5% w/w, less than 3% w/w, less than 1.5% w/w, less than 1% w/w, or less than 0.5% w/w.

Examples of the fatty acid include but are not limited to coconut acid, isostearic acid, myristic acid, oleic acid, ricinoleic acid, stearic acid, undecylenic acid, or combinations thereof.

An example of a suitable fatty acid may be a monounsaturated omega-9 fatty acid, such as oleic acid (CAS Number 112-80-1, available from First Chemical Works, Zhongzheng District, Taipei City, Taiwan, R.O.C.). In one or more embodiments, oleic acid is in a range with a lower limit of any of 0.1% w/w, 0.2% w/w, 0.3% w/w, 0.4% w/w, 0.5% w/w, 0.6% w/w, 0.7% w/w, 0.8% w/w, or 0.9% w/w, and an upper limit of any of 1.2% w/w, 1.4% w/w, 1.6% w/w, 1.8% w/w, 2.0% w/w, 2.2% w/w, 2.4% w/w, 2.6% w/w, 2.8% w/w, 3.0% w/w, 3.2% w/w, 3.4% w/w, 3.6% w/w, 3.8% w/w, 4.0% w/w, 4.2% w/w, or 4.4% w/w, where any lower limit can be used in combination with any upper limit.

The aqueous system is a mixture of substances that contribute to an aqueous phase of the pharmaceutical composition. The aqueous system may include water, an aminopolycarboxylic acid and/or a salt thereof, a polyacrylic acid, a polyol, and a conjugated acid compound.

In one or more embodiments, water is in a range with a lower limit of any of 35% w/w, 40% w/w, 45% w/w, 50% w/w, 55% w/w, 60% w/w, 65% w/w, or 70% w/w, and an upper limit of any of 80% w/w, 85% w/w, or 90% w/w, where any lower limit can be used in combination with any upper limit.

The aminopolycarboxylic acid and/or the salt thereof is a water-soluble monoamine, diamine, triamine, or tetramine, with two or more carboxylic acid functional groups. The salt thereof may be an alkali salt. The metal in the alkali salt may include sodium, potassium, other suitable metal, or a combination thereof.

An example of a suitable aminopolycarboxylic acid and/or a salt thereof includes but is not limited to ethylenediaminetetraacetic acid (EDTA) and/or a sodium salt thereof, such as disodium EDTA (CAS Number 6381-92-6, available from ACROS Organics, Fair Lawn, New Jersey, U.S.A.).

In one or more embodiments, the aminopolycarboxylic acid and/or the salt thereof, such as disodium EDTA, is in a range with a lower limit of any of 0.002% w/w. 0.004% w/w, 0.006% w/w, or 0.008% w/w, and an upper limit of any of 0.012% w/w. 0.014% w/w, 0.016% w/w, 0.018% w/w, 0.02% w/w, 0.03% w/w, 0.04% w/w, 0.05% w/w, 0.1% w/w, or 1% w/w, where any lower limit can be used in combination with any upper limit.

The polyacrylic acid includes acrylic acid subunits and may be a homopolymer or a crosslinked polymer.

An example of a suitable polyacrylic acid includes but is not limited to Carbopol® polymer (such as CAS Number 9003-01-4, available from Lubrizol Pharmaceuticals, Wickliffe, Ohio, U.S.A., but not limited thereto). In one or more embodiments, the polyacrylic acid, such as Carbopol® polymer, is in a range with a lower limit of any of 0.05% w/w, 0.1% w/w, 0.15% w/w, 0.2% w/w, 0.25% w/w, 0.3% w/w, 0.35% w/w, 0.4% w/w, or 0.45% w/w, and an upper limit of any of 0.55% w/w, 0.6% w/w, 0.65% w/w, 0.7% w/w, 0.75% w/w, 0.8% w/w, 0.85% w/w, 0.9% w/w, 0.95% w/w, 1% w/w, 1.5% w/w, or 2% w/w, where any lower limit can be used in combination with any upper limit.

The polyol is an organic compound that includes multiple hydroxyl groups. The polyol may be a diol, triol, tetrol, pentol, hexol, heptol, octal, nonal, or decol. The polyol may include 2 to 20 carbons. There may be one carbon per alcohol on the polyol. For example, when the polyol is a diol it may be glycol, and when the polyol is a triol it may be glycerol.

An example of a suitable polyol includes but is not limited to glycerol (CAS Number 56-81-5, available from J. T. Baker, Avantor Inc., Radnor, Pennsylvania, U.S.A.). In one or more embodiments, the glycerol is in a range with a lower limit of any of 0.01% w/w, 0.05% w/w, or 1% w/w, and an upper limit of any of 2% w/w, 2.5% w/w, 3% w/w, 3.5% w/w, 4% w/w, 4.5% w/w, or 5% w/w, where any lower limit can be used in combination with any upper limit.

The conjugated acid compound may be from 3 to 20 carbons and includes at least one conjugated acid, which is an α,β-unsaturated acid.

An example of a suitable conjugated acid compound includes but is not limited to sorbic acid (CAS Number 110-44-1, available from First Chemical Works, Zhongzheng District, Taipei City, Taiwan, R.O.C.). In one or more embodiments, the conjugated acid compound, such as sorbic acid, is in a range with a lower limit of any of 0.01% w/w, 0.05% w/w, 0.1% w/w, or 0.15% w/w, and an upper limit of any of 0.25% w/w, 0.3% w/w, 0.35% w/w, 0.4% w/w, 0.45% w/w, 0.5% w/w, 0.55% w/w, 0.6% w/w, 0.65% w/w, 0.7% w/w, 0.75% w/w, 0.8% w/w, 0.85% w/w, 0.9% w/w, 0.95% w/w, or 1% w/w, where any lower limit can be used in combination with any upper limit.

Other components in the pharmaceutical composition include one or more neutralizing agent. The neutralizing agent increases the pH of the pharmaceutical composition that includes compounds having acid functional groups.

An example of a suitable neutralizing agent includes but is not limited to triethanolamine (CAS Number 102-71-6, available from Meru Chem Pvt. Ltd., Mumbai, India). In one or more embodiments, the neutralizing agent, such as triethanolamine, is in a range with a lower limit of any of 0.05% w/w, 0.1% w/w, 0.15% w/w, 0.2% w/w, 0.25% w/w, 0.3% w/w, 0.35% w/w, 0.4% w/w, or 0.45% w/w, and with an upper limit of any of 0.55% w/w, 0.6% w/w, 0.65% w/w, 0.7% w/w, 0.75% w/w, 0.8% w/w, 0.85% w/w, 0.9% w/w, 0.95% w/w, or 1% w/w.

Without wanting to be bound by any theory, the addition of the neutralizing agent to the pharmaceutical composition may provide, among other things, a gelling effect due to the pH increase of the overall pharmaceutical composition. The cream consistency (and one or more organoleptic properties) of the pharmaceutical composition may result from said gelling effect.

### Method of Preparing Pharmaceutical Composition

The pharmaceutical composition of one or more embodiments is prepared by steps of mixing, homogenizing, and neutralizing.

The mixing includes mixing a first part and a second part together. The first part includes the aqueous system of one or more embodiments. The second part includes a mixture of the API, the oil solvent system, the surfactant system, and at least one other ingredient (such as cetostearyl alcohol). The initial mixing does not include addition of triethanolamine.

To prepare the aqueous system, the components of the aqueous system according to one or more embodiments are measured on a balance. The components of the aqueous system are then added into a beaker or suitable container to form a mixture. The mixture is stirred for 3-4 hours and is heated at a temperature of 60 to 70° C. until the components (excipients) of the aqueous system are dissolved.

To prepare the mixture of the API, the oil solvent system, the surfactant system, and at least one other ingredient, the components thereof according to one or more embodiments are measured on a balance. The components are then added into a beaker or suitable container to form a mixture. The mixture is stirred for 3-4 hours and is heated at a temperature of 60 to 70° C. until the components (excipients) of the API, the oil solvent system, the surfactant system, and at least one other ingredient are dissolved.

To prepare the emulsion of pharmaceutical composition, the first part and the second part are mixed together. The first part (aqueous system) is transferred or poured into the mixture of the second part (the API, the oil solvent system, the surfactant system, and the at least one other ingredient). The mixture of the first part and the second part is stirred to homogenize at about 4,500 revolutions per minute (rpm) on a homogenizer for 1 minute at a temperature of 60 to 70° C. The homogenizing forms an emulsion of the pharmaceutical composition.

To prepare the cream formulation of the emulsified pharmaceutical composition, triethanolamine according to one or more embodiments is added, while homogenization continues at 4,500 rpm. After removing from the heat source, this neutralizing by addition of triethanolamine proceeds with homogenization at 4,500 rpm until the temperature reaches below 40° C. Typically, the neutralizing step continues for around 4-6 minutes. The pharmaceutical composition is set aside for storage in a cool, dark place.

### EXAMPLES

Fourteen (14) pharmaceutical compositions were prepared, which are formulations that are represented by the identifiers F2 to F15, as shown in Table 1 (percents in weight by weight, or % w/w represents the weight of the component compared to the overall weight of the pharmaceutical composition).

TABLE 1
Pharmaceutical compositions of formulations F2-F15

| Compositions (%, w/w) | F2 | F3 | F4 | F5 | F6 | F7 | F8 |
|---|---|---|---|---|---|---|---|
| Water (%) | 55.04 | 50.74 | 55.19 | 84.17 | 72.59 | 73.59 | 68.19 |
| Disodium EDTA (%) | 0.01 | 0.01 | 0.01 | 0.01 | 0.01 | 0.01 | 0.01 |
| Carbopol (%) | 0.80 | 0.80 | 1.00 | 0.30 | 0.55 | 0.55 | 0.55 |
| Glycerol (%) | 1.50 | 1.50 | 1.50 | 1.50 | 1.50 | 1.50 | 1.50 |
| Sorbic acid (%) | 0.20 | 0.20 | 0.20 | 0.20 | 0.20 | 0.20 | 0.20 |
| API (Formula I) (%) | 0.20 | 0.20 | 0.10 | 0.02 | 0.10 | 0.10 | 0.10 |
| Isopropyl myristate | 21.00 | 28.00 | 20.00 | 7.00 | 12.00 | 12.00 | 12.00 |
| (IPM) (%) | | | | | | | |
| Cetostearyl alcohol | 6.50 | 6.50 | 6.50 | 3.00 | 6.00 | 3.00 | 6.00 |
| (%) | | | | | | | |
| Benzyl alcohol (%) | 3.00 | 3.00 | 3.00 | 1.00 | 0.50 | 2.50 | 2.50 |
| Diethylene glycol | 3.00 | 0.30 | 3.00 | 0.60 | 0.60 | 0.60 | 3.00 |
| monoethyl ether (%) | | | | | | | |
| Dimethicone (%) | 0.25 | 0.25 | 1.00 | 0.10 | 0.45 | 0.45 | 0.45 |
| Polyoxyl 40 stearate | 5.00 | 5.00 | 5.00 | 1.30 | 3.50 | 3.50 | 3.50 |
| (%) * | | | | | | | |
| Oleic acid (%) | 3.00 | 3.00 | 3.00 | 0.30 | 1.50 | 1.50 | 1.50 |
| Triethanolamine (%) | 0.50 | 0.50 | 0.50 | 0.50 | 0.50 | 0.50 | 0.50 |
| Solvents (%) ** | 27.3 | 31.6 | 27.0 | 8.7 | 13.6 | 15.6 | 18.0 |
| Surfactants (%) *** | 8.0 | 8.0 | 8.0 | 1.6 | 5.0 | 5.0 | 5.0 |
| Solvents/API Ratio | 136.3 | 157.8 | 270.0 | 435.0 | 135.5 | 155.5 | 179.5 |
| Solvents/ | 109.0 | 126.2 | 27.0 | 87.0 | 30.1 | 34.6 | 39.9 |
| Dimethicone Ratio | | | | | | | |

* Reagent grade
** “Solvents” in Table 1 is the sum of isopropyl myristate, benzyl alcohol, diethylene glycol monoethyl ether, and dimethicone.
*** “Surfactants” in Table 1 is the sum of Polyoxyl 40 stearate, and oleic acid.

TABLE 1
Pharmaceutical compositions of formulations F2-F15

| Compositions (%, w/w) | F9 | F10 | F11 | F12 | F13 | F14 | F15 |
|---|---|---|---|---|---|---|---|
| Water(%) | 69.84 | 69.09 | 73.44 | 77.34 | 76.44 | 61.59 | 57.69 |
| Disodium EDTA (%) | 0.01 | 0.01 | 0.01 | 0.01 | 0.01 | 0.01 | 0.01 |
| Carbopol (%) | 0.55 | 0.55 | 0.55 | 0.55 | 0.55 | 0.55 | 0.55 |
| Glycerol (%) | 1.50 | 1.50 | 1.50 | 1.50 | 1.50 | 1.50 | 1.50 |
| Sorbic acid (%) | 0.20 | 0.20 | 0.20 | 0.20 | 0.20 | 0.20 | 0.20 |
| API (Formula I) (%) | 0.10 | 0.10 | 0.10 | 0.10 | 0.10 | 0.10 | 0.10 |
| Isopropyl myristate | 12.00 | 12.00 | 7.00 | 6.00 | 4.00 | 12.00 | 12.00 |
| (IPM) (%) | | | | | | | |
| Cetostearyl alcohol | 6.00 | 6.00 | 6.00 | 4.00 | 6.00 | 4.00 | 6.00 |
| Benzyl alcohol (%) | 2.50 | 2.50 | 3.00 | 3.00 | 0.50 | 2.50 | 2.50 |
| Diethylene glycol | 0.60 | 0.60 | 1.00 | 0.20 | 0.20 | 0.60 | 9.00 |
| monocthyl ether (%) | | | | | | | |
| Dimethicone (%) | 1.20 | 0.45 | 0.20 | 0.10 | 3.50 | 0.45 | 0.45 |
| Polyoxyl 40 stearate | 3.50 | 5.00 | 3.50 | 3.50 | 3.50 | 9.50 | 5.00 |
| (%) * | | | | | | | |
| Oleic acid (%) * | 1.50 | 1.50 | 3.00 | 3.00 | 3.00 | 6.50 | 4.50 |
| Triethanolamine (%) | 0.50 | 0.50 | 0.50 | 0.50 | 0.50 | 0.50 | 0.50 |
| Solvents (%) ** | 16.3 | 15.6 | 11.2 | 9.3 | 8.2 | 15.6 | 24.0 |
| Surfactants (%) *** | 5.0 | 6.5 | 6.5 | 6.5 | 6.5 | 16.0 | 9.5 |
| Solvents/API Ratio | 163.0 | 155.5 | 112.0 | 93.0 | 82.0 | 155.5 | 239.5 |
| Solvents/ | 13.6 | 34.6 | 56.0 | 93.0 | 2.3 | 36.0 | 53.3 |
| Dimethicone Ratio | | | | | | | |

* Reagent grade
** “Solvents” in Table 1 is the sum of isopropyl myristate, benzyl alcohol, diethylene glycol monoethyl ether, and dimethicone.
*** “Surfactants” in Table 1 is the sum of Polyoxyl 40 stearate, and oleic acid.

Formulations F2 to F15 were developed and evaluated for API (Formula I) stability and composition stability.

Formulations that provide sufficient API stability and composition stability are considered pharmaceutical compositions.

To prepare a formulation, an aqueous system was first prepared. The appropriate amount of disodium EDTA, Carbopol®, glycerol, sorbic acid, and water were measured on a balance (Sartorius™) and added as a mixture into a beaker. The beaker was placed in a water bath and the mixture was stirred on a stirring hot plate (Corning® PC-420D) and heated at a temperature of 60 to 70° C. until the excipients dissolved, for 3 to 4 hours. The resulting mixture, the aqueous system, was kept at 60 to 70° C. prior to emulsification.

Second, a mixture of including an API, an oil solvent system, and surfactant system was prepared. The appropriate amount of API (Formula I), isopropyl myristate (IPM), cetostearyl alcohol, benzyl alcohol, diethylene glycol monoethyl ether, dimethicone, polyoxyl 40 stearate, and oleic acid were measured on a balance (Sartorius™) and added as a mixture into a 100 milliliter (mL) glass bottle. The glass bottle was placed in a water bath and the mixture was stirred on a stirring hot plate (Corning® PC-420D) and heated at a temperature of 60 to 70° C. until the excipients were dissolved, for 3 to 4 hours. The resulting mixture, of API, oil solvent system, and surfactant system, was kept at 60 to 70° C. prior to emulsification.

Third, a homogenizer (Omni Programmable Digital Homogenizer, or Omni PDH, OMNI International™) was set to 4,500 revolutions per minute (rpm). The aqueous system was transferred into the mixture of the API, oil solvent system, and surfactant system. The homogenizer emulsified the mixture of combined solutions at 4,500 rpm for 1 minute at 60-70° C.

Last, triethanolamine was added into the mixture of the combined solutions, while the homogenization continued at 4,500 rpm. Upon addition of triethanolamine, the mixture was cooled in a room temperature water bath for 4-6 minutes, to allow the mixture to reach a temperature below 40° C. The resulting formulation was set aside and stored in a cool, dark place.

Formulations F2 to F15 were tested for chemical and physical stability. F2 to F15 were analyzed by light reflection and absorption, including polarized light microscopy and the chromatography with a photo diode array (PDA) to detect ultraviolet and visible light (UV-vis) regions.

### Polarized Light Microscopy Analysis

Polarized light microscopy was used to analyze formulations F2 to F15, with a polarized light microscope. The procedure is as follows. A sample of the formulation (one sample for each of F2 to F15) was set at room temperature (15-20° C.) for 1 day and then examined under a polarized light microscope to observe the presence or absence of API crystal formation (of Formula I). The absence of API crystal formation indicates sufficient solubility and physical stability. The presence of API crystal formation indicates insufficient solubility, insufficient physical stability, or both.

FIG. 2 shows the polarized light microscopy of sample F2. No API crystals were found, indicating sufficient solubility and physical stability. The light on the background is from the light reflection of oil droplets (noise).

FIG. 3 shows the polarized light microscopy of sample F3. No API crystals were found, indicating sufficient solubility and physical stability. The light on the background is from the light reflection of oil droplets (noise).

FIG. 4 shows the polarized light microscopy of sample F4. No API crystals were found, indicating sufficient solubility and physical stability. The light on the background is from the light reflection of oil droplets (noise).

FIG. 5 shows the polarized light microscopy of sample F5. No API crystals were found, indicating sufficient solubility and physical stability. The light on the background is from the light reflection of oil droplets (noise).

FIG. 6 shows the polarized light microscopy of sample F6. No API crystals were found, indicating sufficient solubility and physical stability. The light on the background is from the light reflection of oil droplets (noise).

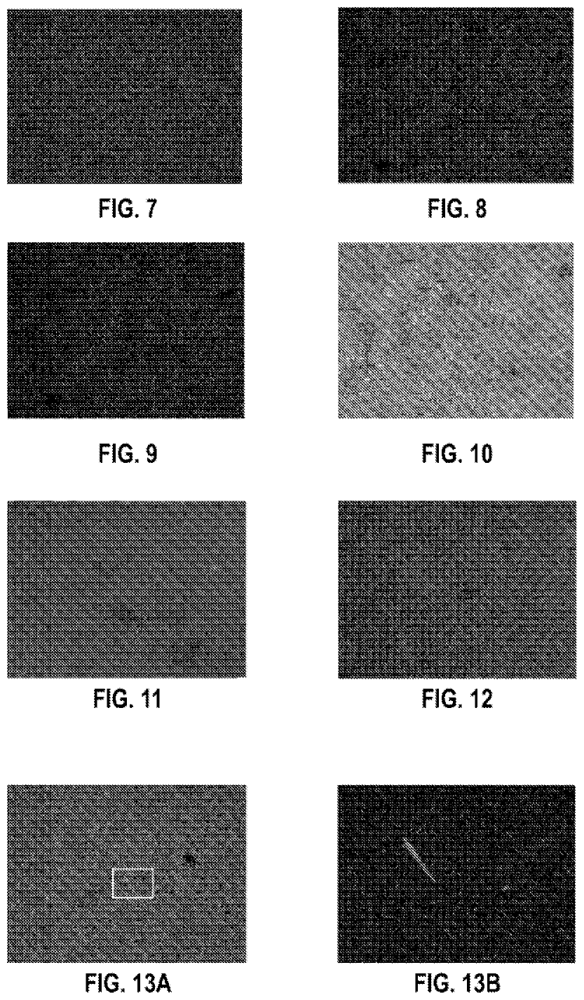

FIG. 7 shows the polarized light microscopy of sample F7. No API crystals were found, indicating sufficient solubility and physical stability. The light on the background is from the light reflection of oil droplets (noise).

FIG. 8 shows the polarized light microscopy of sample F8. No API crystals were found, indicating sufficient solubility and physical stability. The light on the background is from the light reflection of oil droplets (noise).

FIG. 9 shows the polarized light microscopy of sample F9. No API crystals were found, indicating sufficient solubility and physical stability. The light on the background is from the light reflection of oil droplets (noise).

FIG. 10 shows the polarized light microscopy of sample F10. No API crystals were found, indicating sufficient solubility and physical stability. The light on the background is from the light reflection of oil droplets (noise).

FIG. 11 shows the polarized light microscopy of sample F11. No API crystals were found, indicating sufficient solubility and physical stability. The light on the background is from the light reflection of oil droplets (noise).

FIG. 12 shows the polarized light microscopy of sample F12. No API crystals were found, indicating sufficient solubility and physical stability. The light on the background is from the light reflection of oil droplets (noise).

FIG. 13A shows the polarized light microscopy of sample F13. FIG. 13A shows a white box surround, where FIG. 13B shows a zoomed-in version of this surround. FIGS. 13A and 13B show that API crystals were found with polarized light and an acicular (needle) shape. The polarized light reflecting off the API crystal is a yellow or yellow-orange light (shown as white or gray in FIGS. 13A and 13B) that is set against a black background with small white lights, which is from the light reflection of oil droplets (noise).

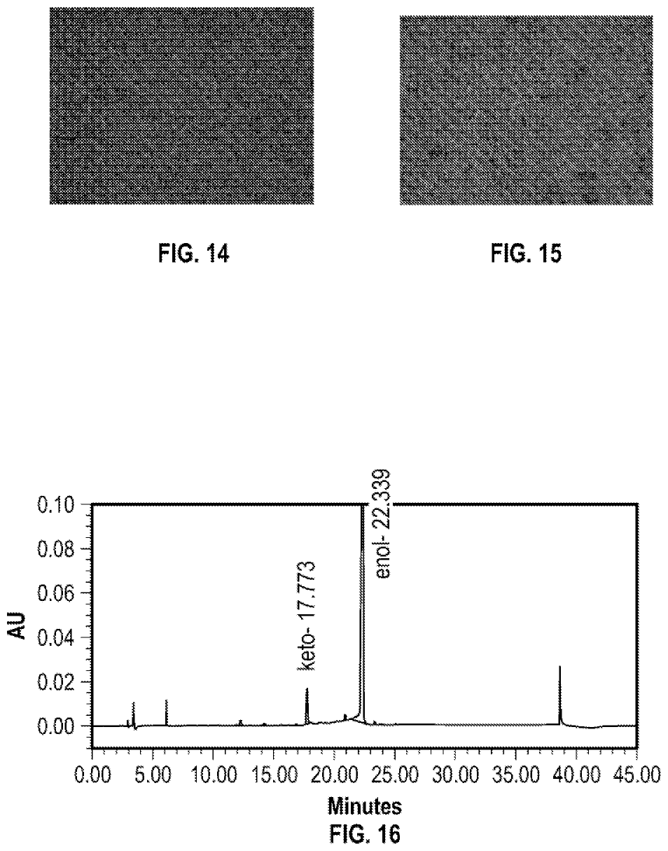

FIG. 14 shows the polarized light microscopy of sample F14. No API crystals were found, indicating sufficient solubility and physical stability. The light on the background is from the light reflection of oil droplets (noise).

FIG. 15 shows the polarized light microscopy of sample F15. No API crystals were found, indicating sufficient solubility and physical stability. The light on the background is from the light reflection of oil droplets (noise).

### HPLC Analysis

The procedure for HPLC (high performance liquid chromatography) analysis is as follows. A 10 gram (g) sample of formulation (one sample for each of F2 to F15) was heated to a temperature of 50° C. for 6 days. The purity of the formulation, including chemical stability of the API (Formula I), was analyzed. Control tests accompanied the stability tests, using the same procedure (one sample for each of F2 to F15) at a temperature of 5° C. (refrigerated) for 6 days.

FIG. 16 shows a chromatogram (HPLC analysis, 350 nm) of the stability testing of sample F7 (50° C. for 6 days), with tautomer retention time (“RT”) peaks at 17.78 minutes (min.) and 22.345 min., respectively.

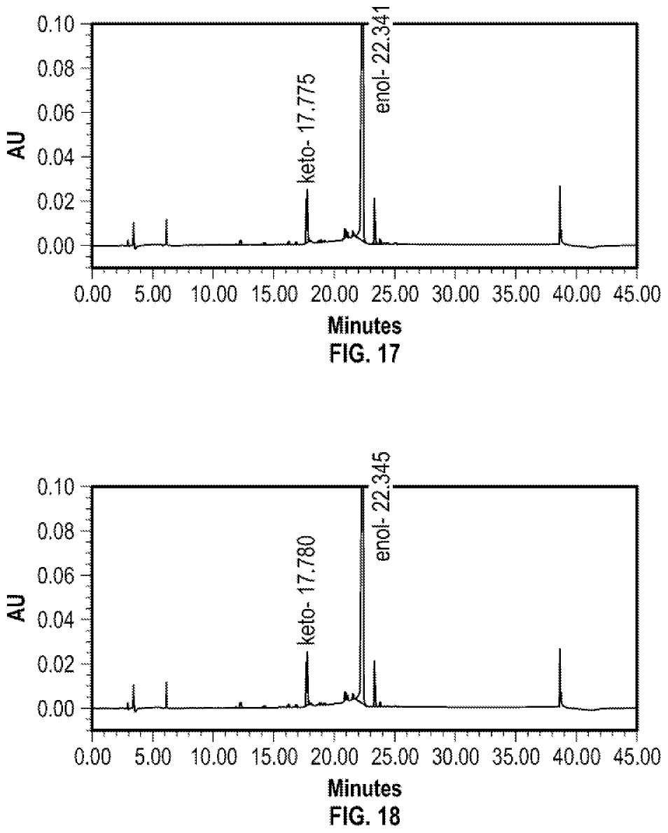

FIG. 17 shows a chromatogram (HPLC analysis, 350 nm) of the stability testing of sample F14 (50° C. for 6 days), with tautomer RT peaks at 17.775 min. and 22.341 min., respectively.

FIG. 18 shows a chromatograph (HPLC analysis, 350 nm) of the stability testing of sample F15 (50° C. for 6 days), with tautomer RT peaks at 17.780 min. and 22.345 min., respectively.

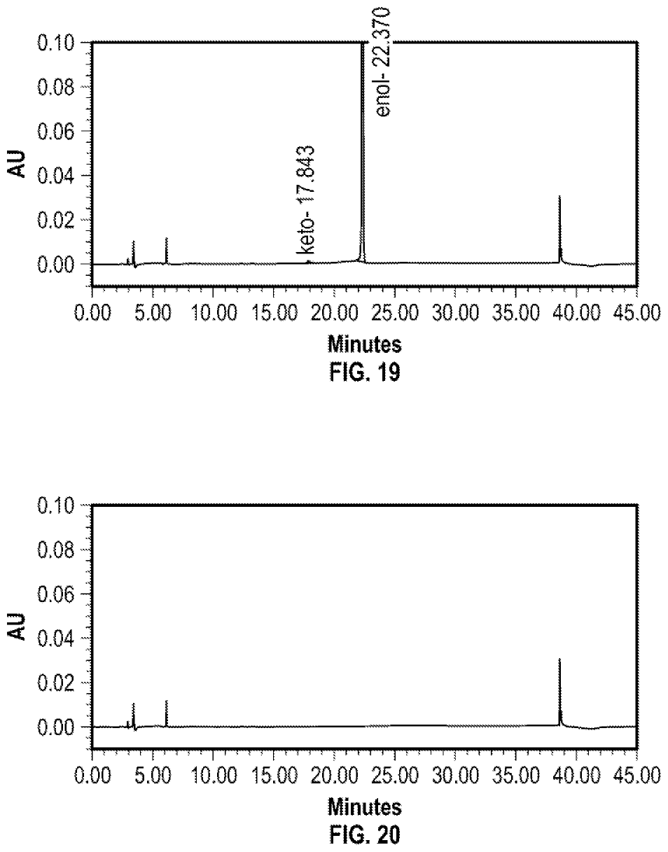

FIG. 19 shows a chromatograph (HPLC analysis, 350 nm) of the standard solution of API (Formula I) as a control.

FIG. 20 shows a chromatograph (HPLC analysis, 350 nm) of the blank solution (diluent) with solvent/diluent only (no API, Formulation, or any other excipients).

Without being bound by theory, it is believed that the API (Formula I) tautomerizes, including an enol and keto form. The keto form of the API (Formula I) may elute earlier (about 17.78 min), and the enol form of the API (Formula I) may elute later (about 22.34 to 22.35 min), when using this HPLC method. The enol form of the tautomer may be stabilized by conjugation as well as intramolecular hydrogen bonding between the enol proton and the ketone oxygen that are at positions 1,3 to each other (Formula I shows the keto form of the tautomer, including a 1,3-diketone), within the parameters of the HPLC analysis.

### Solubility of API in Presence of Aqueous System

The API of Formula I is a hydrophobic molecule that is practically insoluble in an aqueous phase, in this case the aqueous system (Reference world wide web address “solubilityofthings.com/levels-of-solubility”). When the API of Formula I is in the aqueous system, it forms crystals and precipitates out of solution. To investigate a compatible matrix that allows greater Formula I solubility, various systems containing Formula I were stored at room temperature for 24 hours and were examined under a polarized light microscope to observe the presence or absence of Formula I crystal precipitation.

It was unexpectedly found that, of the various systems, a presence of a high concentration of dimethicone leads to the precipitation of Formula I.

For example, Formulation F13 includes the lowest amount of total solvents when compared with Formulations F9, F11, and F12. Crystals of API (Formula I) were observed in F13 after 24 hours at room temperature, and were not observed in F9, F11, or F12 as a comparison. The “total solvents” in this instance are the sum of isopropyl myristate, benzyl alcohol, diethylene glycol monoethyl ether, and dimethicone. The solvent-to-API ratio is the “total solvents” compared to the weight % of the API (Formula I) in a formulation. The solvent-to-dimethicone ratio is the “total solvents” compared to the weight % of the dimethicone in a formulation.

TABLE 2
Solubility comparison of F3, F9, F11, F12, and F13

| Compositions (%, w/w) | F3 | F9 | F11 | F12 | F13 |
|---|---|---|---|---|---|
| Total Solvents (%) | 31.6 | 16.3 | 11.2 | 9.3 | 8.2 |
| Solvent-to-API Ratio | 158.0 | 163.0 | 112.0 | 93.0 | 82.0 |
| Solvent-to-dimethicone Ratio | 126.2 | 13.6 | 56.0 | 93.0 | 2.3 |
| API Precipitation | No | No | No | No | Yes |

Although F13 contains the lowest amount of solvents (% w/w) and the lowest solvent-to-API ratio, F12 includes a similar amount of solvents and solvent-to-API ratio, when compared to F13. The rapid API precipitation observed in F13 may result from a low amount of solvents and also from a low solvent-to-dimethicone ratio. The results indicated that the greater amount of dimethicone may cause precipitation of Formula I from a formulation.

Formulation F13, which contains the lowest amount of solvents in any of F2-F15, was observed with API crystals, indicating that it may contain a matrix that favors API precipitation. However, F12 also contains a similar range of solvents but API does not precipitate from F12. Thus, the presence of dimethicone appears to unexpectedly decrease the solubility of API in the formulation. Additionally, a wide range of total solvents appear to be providing sufficient solubility of API in formulations (Table 2), depending on the concentration of Formula I in the formulation. It is difficult to determine a range or boundary for an amount of solvents suitable with different concentrations of Formula I in a formulation. Therefore, the solvent-to-API ratio was used to clarify and to set a limit of total solvents that allow sufficient Formula I solubility. In summary, the solubility results of Formula I in the Formulations F9, F11, F12, and F13 indicate that Formula I readily precipitates when the solvent-to-API ratio is less than 82.0 (82:1) and when the solvent-to-dimethicone ratio is less than 2.3 (2.3:1). Thus, it was unexpectedly found that these two factors (solvent-to-API ratio of 82 or greater, and solvent-to-dimethicone ratio of 2.3 or greater) may provide sufficient stability of the API of Formula I.

### API Solubility in Different Dimethicone

Next, different types of dimethicone having viscosities, 4.6 centistokes (cSt) and 500 cSt, respectively, were evaluated to determine whether the different dimethicone types would have a similar effect on API crystallization (Table 3). The API's solubility was examined in cream formulations with dimethicone concentrations ranging from 1.20% w/w to 7.00% w/w.

TABLE 3
Formulations prepared for dimethicone assessment

| | D1 | D2 | D3 | D4 | DS |
|---|---|---|---|---|---|
| | Design | | | | |
| | Different types | | | Dimethicone at | |
| Compositions (%, w/w) | of dimethicone | | | preferred conc. | |
| Water (%) | 76.44 | 76.44 | 72.94 | 69.84 | 69.84 |
| Disodium EDTA (%) | 0.01 | 0.01 | 0.01 | 0.01 | 0.01 |
| Carbopol (%) | 0.55 | 0.55 | 0.55 | 0.55 | 0.55 |
| Glycerol (%) | 1.50 | 1.50 | 1.50 | 1.50 | 1.50 |
| Sorbic acid (%) | 0.20 | 0.20 | 0.20 | 0.20 | 0.20 |
| API (Formula I) (%) | 0.10 | 0.10 | 0.10 | 0.10 | 0.10 |
| Isopropyl myristate (%) | 4.00 | 4.00 | 4.00 | 12.00 | 12.00 |
| Cetostearyl alcohol (%) | 6.00 | 6.00 | 6.00 | 6.00 | 6.00 |
| Benzyl alcohol (%) | 0.50 | 0.50 | 0.50 | 2.50 | 2.50 |
| Diethylene glycol | 0.20 | 0.20 | 0.20 | 0.60 | 0.60 |
| monoethyl ether (%) | | | | | |
| Dimethicone 500 cSt (%) | 3.50 | | | 1.20 | |
| Dimethicone 4.6 cSt (%) | | 3.50 | 7.00 | | 1.20 |
| Polyoxyl 40 stearate * | 3.50 | 3.50 | 3.50 | 3.50 | 3.50 |
| Oleic acid (%) * | 3.00 | 3.00 | 3.00 | 1.50 | 1.50 |
| Triethanolamine (%) | 0.50 | 0.50 | 0.50 | 0.50 | 0.50 |
| Solvent (%) ** | 8.2 | 8.2 | 11.7 | 16.3 | 16.3 |
| Surfactant (%) *** | 6.50 | 6.50 | 6.50 | 5.00 | 5.00 |
| Solvent/API Ratio | 82.0 | 82.0 | 117.0 | 163.0 | 163.0 |
| Solvent/Dimethicone Ratio | 2.3 | 2.3 | 1.7 | 13.6 | 13.6 |

* Reagent grade
** “Solvent” in Table 3 is the sum of isopropyl myristate, benzyl alcohol, diethylene glycol monoethyl ether, and dimethicone.
*** “Surfactant” in Table 3 is the sum of Polyoxyl 40 stearate and oleic acid.

When the dimethicone concentration in formulations was 1.20% w/w, no crystallization was observed regardless of the dimethicone types. On the other hand, API crystallization was observed under polarized light microscopy at the concentration of 3.50% w/w (Table 4).

Additionally, API crystallization was observed in cream formulation with 4.6 cSt dimethicone at 7.00% w/w event when with the solvent to API ratio as 117 (Table 4). The results indicated that the amount of dimethicone is a critical factor that leads to API solubility. Exceedingly high concentration (as high as 7% w/w) led to API crystallization regardless of the high Solvent to API ratio. Thus, the dimethicone should be lower than 3.5% w/w.

TABLE 4
Polarized light microscopic assessment of API crystallization

| Compositions (%, w/w) | D1 | D2 | D3 | D4 | D5 |
|---|---|---|---|---|---|
| Dimethicone (%) | 3.50 | 3.50 | 7.00 | 1.20 | 1.20 |
| Dimethicone | 500 | 4.6 | 4.6 | 500 | 4.6 |
| viscosity (cSt) | | | | | |
| Solvent (%) * | 8.2 | 8.2 | 11.7 | 16.3 | 16.3 |
| Solvent to API Ratio | 82.0 | 82.0 | 117.0 | 163.0 | 163.0 |
| Solvent to | 2.3 | 2.3 | 1.7 | 13.6 | 13.6 |
| Dimethicone Ratio | | | | | |
| API | Yes | Yes | Yes | No | No |
| crystallization | (FIG. | (FIG. | (FIG. | (FIG. | (FIG. |
| | 6-1, | 6-1, | 6-1, | 6-1, | 6-1, |
| | D1) | D2) | D3) | D4) | D5) |

* “Solvent” in Table 4 is the sum of isopropyl myristate, benzyl alcohol, diethylene glycol monoethyl ether, and dimethicone.

### Polarized Light Microscopy Analysis of Formulation D1 to D5

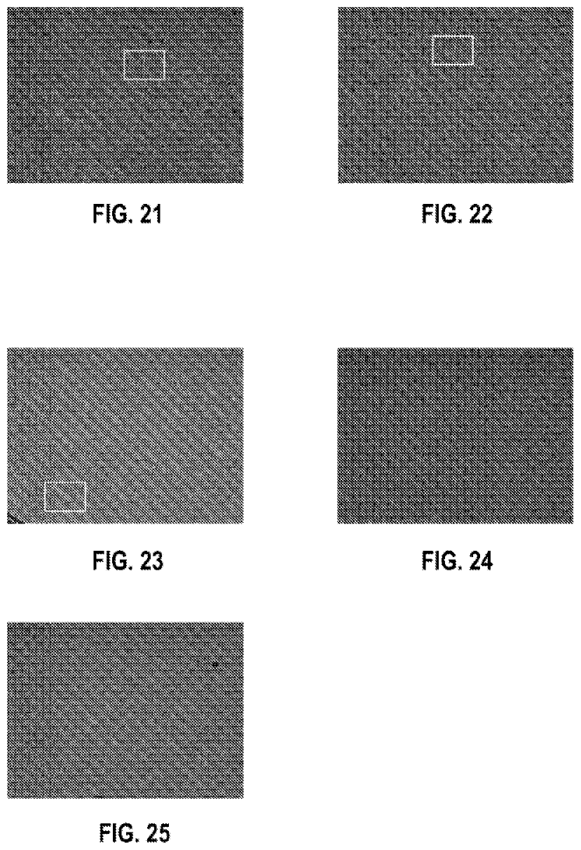

FIG. 21 shows the polarized light microscopy of sample D1. API crystals were found with polarized light and an acicular (needle) shape, indicated with a white box surround (there are more API crystals in FIG. 21 than shown in the white box surround). The polarized light reflecting off the API crystal is a yellow or yellow-orange light (shown as white or gray in FIGS. 21 to 23) that is set against a black background with small white lights, which is from the light reflection of oil droplets (noise).

FIG. 22 shows the polarized light microscopy of sample D2. API crystals were found with polarized light and an acicular (needle) shape, indicated with a white box surround (there are more API crystals in FIG. 22 than shown in the white box surround).

FIG. 23 shows the polarized light microscopy of sample D3. API crystals were found with polarized light and an acicular (needle) shape, indicated with a white box surround (there are more API crystals in FIG. 23 than shown in the white box surround).

FIG. 24 shows the polarized light microscopy of sample D4. No API crystals were found, indicating sufficient solubility and physical stability. The light on the background is from the light reflection of oil droplets (noise).

FIG. 25 shows the polarized light microscopy of sample D5. No API crystals were found, indicating sufficient solubility and physical stability. The light on the background is from the light reflection of oil droplets (noise).

### Formulation Purity and Impurities

Purity and total impurities (%) of Formula I in Formulations F2 to F15 are shown in Table 5.

In this instance, “impurity” or “impurities” is defined as loss of API % purity (by weight) Without wanting to be bound by any theory, it is assumed that the loss of API % purity, and thus impurities, may include the formation of other compounds besides the API as is known in the art of pharmaceutical formulations and that these compounds, where present, have a similar extinction coefficient at 350 nm as the API (for purposes of calculation).

TABLE 5
Purity and impurities of Formulations F2-F15

| | 5° C. for 6 days (Control) | | 50° C. for 6 days | |
|---|---|---|---|---|
| | | Total | | Total |
| | Purity (%)* | impurities(%) | Purity (%)* | impurities(%) |
| F2 | 99.66 | 0.34 | 96.96 | 3.04 |
| F3 | 99.74 | 0.26 | 98.04 | 1.96 |
| F4 | 99.67 | 0.33 | 97.79 | 2.21 |
| F5 | 99.31 | 0.69 | 97.91 | 2.09 |
| F6 | 99.66 | 0.34 | 97.90 | 2.10 |
| F7 | 99.71 | 0.29 | 97.78 | 2.22 |
| F8 | 99.43 | 0.57 | 96.10 | 3.90 |
| F9 | 99.56 | 0.44 | 97.70 | 2.30 |
| F10 | 99.40 | 0.60 | 97.48 | 2.52 |
| F11 | 99.37 | 0.63 | 96.59 | 3.41 |
| F12 | 99.38 | 0.62 | 97.33 | 2.67 |
| F13 | 99.27 | 0.73 | 96.91 | 3.09 |
| F14 | 98.77 | 1.23 | 91.35 | 8.65 |
| F15 | 98.69 | 1.31 | 91.42 | 8.58 |

*Purity is the area percentage of the sum of API keto and enol tautomers.

As previously described, the formulations were placed at 50° C. for 6 days and were analyzed by HPLC. In this series of experiments, a formulation with greater than 5% total impurities provided under the testing conditions as described were considered unsuitable. Thus, sufficient stability may be further defined by a 5% total impurities or less according to the results of the stability testing.

Among the formulations, F14 and F15 provided greater than 5% total impurities after stability testing as shown in Table 5. Formulations F2 to F13 provided 5% or less total impurities after stability testing as shown in Table 5. Among the group of F2 to F13, the greatest impurity level observed was F8, at 3.90%. Thus, stability of the API (chemical stability) of Formula I in Formulations F2 to F13 is superior to stability of the API of Formula I in Formulations F14 and F15. F14 contained the greatest amount of surfactants (polyoxyl 40 stearate and oleic acid), unexpectedly resulting in API instability when the surfactant system exceeded 16% (w/w) of the formulation. However, it is noted that the API rapidly precipitated from Formulation F13 in previous solubility tests. In this series of experiments (HPLC to determine purity and impurities), an aliquot of each Formulation was dissolved before running a sample on HPLC, as is standard procedure in the art. Thus, these HPLC experiments provide purity and impurity information regarding the API. The representative HPLC chromatogram at 350 nm of F2, F14, and F15 (from stability testing at 50° C. for 6 days) is shown in FIGS. 16-18.

TABLE 6
Purity and impurities of select formulations, compared to composition elements

| | 5° C. for 6 days | | | | Composition | |
|---|---|---|---|---|---|---|
| | (Control) | | | | | Diethylene |
| | | Total | 50° C. for 6 days | | | glycol |
| | Purity | impurities | Purity | Total | Surfactants | monoethyl |
| | (%)* | (%) | (%)* | impurities (%) | (%)** | ether (%) |
| F3 | 99.74 | 0.26 | 98.04 | 1.96 | 8.0 | 0.3 |
| F4 | 99.67 | 0.33 | 97.79 | 2.21 | 8.0 | 3.0 |
| F5 | 99.31 | 0.69 | 97.91 | 2.09 | 1.6 | 0.6 |
| F7 | 99.71 | 0.29 | 97.78 | 2.22 | 5.0 | 0.6 |
| F14 | 98.77 | 1.23 | 91.35 | 8.65 | 16.0 | 0.6 |
| F15 | 98.69 | 1.31 | 91.42 | 8.58 | 9.5 | 9.0 |

*Purity is the area percentage of the sum of API keto and enol tautomers.
**“Surfactant” in Table 6 is the sum of Polyoxyl 40 stearate and oleic acid.

As shown in Table 6, the surfactant system (total amount of surfactants) in F15 was 9.5%, which was similar to F4 where the total amount of surfactants was 8%. However, the total impurities observed in F15 (8.58%) were much greater than the total impurities observed in F4 (2.21%). When comparing the compositions of F4 and F15 from Table 6, F15 had a greater amount of diethylene glycol monoethyl ether. Thus, an amount of diethylene glycol monoethyl ether in the formulation may be a contributing factor to sufficient stability of the API. A well-known penetration enhancer for topical drugs, diethylene glycol monoethyl ether provides good solubility for the API (Formula I). However, instability of the API in greater amounts of diethylene glycol monoethyl ether may indicate that further addition of solvents and enhancers for the API (Formula I) is difficult.

Formulation F8 had the greatest total impurities of F2 to F12. However, the differences of total impurities between F2 to F12 were less than 5%. This narrow range of total impurities between F2 to F12 may be due to an effect that each component has on the instability of API (Formula I).

Concentration of Non-Ionic Surfactant that Affects API Stability

Formulations using various types of non-ionic surfactants at high, medium and low concentrations, 25% w/w, 9.50% w/w, and 5.00% w/w, were further evaluated for API stability (Table 7). Purity and total impurities (%) of API in the formulations S1-S3, S5-S7, and S9-S11 were shown in Table 8. The formulations were placed in a stability chamber and tested for their stability at 50° C. on the 6^{th} day or the 17^{th} day.

TABLE 7
Formulations with various type of non-ionic surfactants

| | S1 | S2 | S3 | S5 | S6 | S7 | S9 | S10 | S11 |
|---|---|---|---|---|---|---|---|---|---|
| | Design | | | | | | | | |
| | Non-ionic surfactants | | | Non-ionic surfactants | | | Non-ionic surfactants | | |
| Compositions (%, w/w) | at 9.50% | | | at 5.0% | | | at 25% | | |
| Water (%) | 65.09 | 65.09 | 65.09 | 67.59 | 67.59 | 67.59 | 54.59 | 54.59 | 54.59 |
| Disodium EDTA (%) | 0.01 | 0.01 | 0.01 | 0.01 | 0.01 | 0.01 | 0.01 | 0.01 | 0.01 |
| Carbopol (%) | 0.55 | 0.55 | 0.55 | 0.55 | 0.55 | 0.55 | 0.55 | 0.55 | 0.55 |
| Glycerol (%) | 1.50 | 1.50 | 1.50 | 1.50 | 1.50 | 1.50 | 1.50 | 1.50 | 1.50 |
| Sorbic acid (%) | 0.20 | 0.20 | 0.20 | 0.20 | 0.20 | 0.20 | 0.20 | 0.20 | 0.20 |
| API (Formula I) (%) | 0.10 | 0.10 | 0.10 | 0.10 | 0.10 | 0.10 | 0.10 | 0.10 | 0.10 |
| Isopropyl myristate | 12.00 | 12.00 | 12.00 | 12.00 | 12.00 | 12.00 | 12.00 | 12.00 | 12.00 |
| (%) | | | | | | | | | |
| Cetostearyl alcohol | 4.00 | 4.00 | 4.00 | 6.00 | 6.00 | 6.00 | 4.00 | 4.00 | 4.00 |
| Benzyl alcohol (%) | 2.50 | 2.50 | 2.50 | 2.50 | 2.50 | 2.50 | 2.50 | 2.50 | 2.50 |
| Diethylene glycol | 0.60 | 0.60 | 0.60 | 0.60 | 0.60 | 0.60 | 0.60 | 0.60 | 0.60 |
| monoethyl ether (%) | | | | | | | | | |
| Dimethicone 100 cSt | 0.45 | 0.45 | 0.45 | 0.45 | 0.45 | 0.45 | 0.45 | 0.45 | 0.45 |
| (%) | | | | | | | | | |
| Polyoxyl 40 stearate | 9.50 | | | 5.00 | | | 25.00 | | |
| (%) * | | | | | | | | | |
| Polysorbate 20 (%) * | | 9.50 | | | 5.00 | | | 25.00 | |
| Polysorbate 80 (%) * | | | 9.50 | | | 5.00 | | | 25.00 |
| Oleic acid (%) * | 3.00 | 3.00 | 3.00 | 3.00 | 3.00 | 3.00 | 3.00 | 3.00 | 3.00 |
| Triethanolamine (%) | 0.50 | 0.50 | 0.50 | 0.50 | 0.50 | 0.50 | 0.50 | 0.50 | 0.50 |
| Solvent (%) ** | 15.6 | 15.6 | 15.6 | 15.6 | 15.6 | 15.6 | 15.6 | 15.6 | 15.6 |
| Surfactant (%) *** | 12.50 | 12.50 | 12.50 | 8.00 | 8.00 | 8.00 | 28.00 | 28.00 | 28.00 |
| Solvent/API Ratio | 155.5 | 155.5 | 155.5 | 155.5 | 155,5 | 155,5 | 155.5 | 155.5 | 155.5 |
| Solvent/Dimethicone | 34.6 | 34.6 | 34.6 | 34.6 | 34.6 | 34.6 | 34.6 | 34.6 | 34.6 |
| Ratio | | | | | | | | | |

* NF or pharmaceutical grades.
** “Solvent” in Table 7 is the sum of isopropyl myristate, benzyl alcohol, diethylene glycol monoethyl ether, and dimethicone.
*** “Surfactant” in Table 7 is the sum of oleic acid and Polyoxyl 40 stearate, Polysorbate 20, or Polysorbate 80.

TABLE 8
Total impurity of API in various concentration
of non-ionic surfactant

| | 6 days | | 17 days | | Non-ionic surfactants | |
|---|---|---|---|---|---|---|
| | 5° C. | 50° C. | 5° C. | 50° C. | % w/w | Types |
| S1 | | | 0.28 | 5.78 | 9.50 | Polyoxyl 40 stearate |
| S2 | | | 0.70 | 5.13 | | Polysorbate 20 |
| S3 | | | 1.17 | 5.04 | | Polysorbate 80 |
| SS | | | 0.35 | 3.19 | 5.00 | Polyoxyl 40 stearate |
| S6 | | | 0.49 | 2.21 | | Polysorbate 20 |
| S7 | | | 0.57 | 2.00 | | Polysorbate 80 |
| S9 | 2.77 | 14.82 | | | 25.00 | Polyoxyl 40 stearate |
| S10 | 1.99 | 10.84 | | | | Polysorbate 20 |
| S11 | 4.90 | 12.12 | | | | Polysorbate 80 |

*Total impurities were the sum of the percentage area of HPLC peaks other than API peak(s) (API peak(s) include keto and enol tautomer peaks).

Formulations with 9.5% w/w and 25% w/w of non-ionic surfactants were observed with insufficient API stability. Additionally, with a much higher concentration of non-ionic surfactants (25% w/w), formulation S9-S11 showed exceedingly high impurities as early as the 6th day. Therefore, the amount of non-ionic surfactant should be limited to be a concentration lower than 9.5% w/w to avoid instability issues.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to =10%, of up to 5%, of up to 2%, of up to 1%, of up to 0.5%, of up to 0.1%, or up to 0.01%.

The term “substantially” as used refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Ranges may be expressed as from about one particular value to about another particular value, inclusive. When such a range is expressed, it is to be understood that another embodiment is from the one particular value to the other particular value, along with all particular values and combinations thereof within the range.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. It is the express intention of the applicant not to invoke 35 U.S.C. § 112(f) for any limitations of any of the claims herein, except for those in which the claim expressly uses the words ‘means for’ together with an associated function.

What is claimed:

1. A pharmaceutical composition suitable for topical application, comprising:
(i) a curcuminoid compound of Formula I and/or a salt thereof as an active pharmaceutical ingredient (API) in a range of from 0.0019% w/w to 0.20% w/w; and 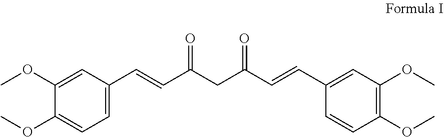
(ii) an oil solvent system in an amount of at least 8% w/w,
wherein % w/w is compared to the overall weight of the pharmaceutical composition.

2. The pharmaceutical composition of claim 1, further comprising:
(iii) a surfactant system in a range of from 1.6% w/w to 16% w/w, said surfactant system comprising a non-ionic surfactant and a fatty acid;
wherein the oil solvent system is in a range of from 89% w/w to 32% w/w, said oil solvent system comprising isopropyl myristate, benzyl alcohol, diethylene glycol monoethyl ether, and dimethicone.

3. The pharmaceutical composition of claim 2,
wherein the non-ionic surfactant is one or more selected from the group consisting of polyethylene glycol ether of cetearyl alcohol, polyoxyethylene alkyl ether, polyethylene glycol ether of cholesterol, polyoxyethylene ether of lanolin alcohol, ethoxylated methyl glucoside, ethoxylated alkyl phenol, polyethylene glycol ether of oleyl alcohol, polyoxyethylene-polyoxypropylene block copolymer, polyoxyethylene fatty acid ester, polyoxyl glyceryl stearate, polyoxyethylene sorbitan monolaurate, polyoxyethylene stearyl ether, polydimethyl siloxane, methyl glucose poly-ester, methyl glucose poly-ether, polyethylene glycol derivative of castor oil, polyethylene glycol derivative of fatty acid ester, and polyethylene glycol derivative of alcohol ester, and combinations thereof; and
wherein the fatty acid is one or more selected from the group consisting of coconut acid, isostearic acid, myristic acid, oleic acid, ricinoleic acid, stearic acid, undecylenic acid, and combinations thereof.

4. The pharmaceutical composition of claim 2, wherein the dimethicone is in an amount less than 3.5% w/w.

5. The pharmaceutical composition of claim 4, wherein the oil solvent system and API have a solvent-to-API ratio of more than 82:1.

6. The pharmaceutical composition of claim 4, wherein the oil solvent system and the dimethicone have a solvent-to-dimethicone ratio of more than 2.3:1.

7. The pharmaceutical composition of claim 2, wherein the surfactant system is in an amount less than 16% w/w.

8. The pharmaceutical composition of claim 2, wherein the fatty acid is in an amount less than 6.5% w/w.

9. The pharmaceutical composition of claim 2, wherein the non-ionic surfactant is in an amount of less than 9.5% w/w.

10. The pharmaceutical composition of claim 2, wherein the diethylene glycol monoethyl ether is less than 9% w/w.
